(19) United States
(12) Patent Application Publication
(10) Pub. No.: US 2025/0303124 A1
(43) Pub. Date: Oct. 2, 2025

(54) VESSEL MODIFICATION USING A THERAPEUTIC AGENT

(71) Applicant: Medtronic, Inc., Minneapolis, MN (US)

(72) Inventors: Carlos H. Lima, Santa Rosa, CA (US); Stefan S. Tunev, Santa Rosa, CA (US)

(21) Appl. No.: 18/851,558

(22) PCT Filed: Mar. 9, 2023
(86) PCT No.: PCT/IB2023/052256

§ 371 Date: Sep. 26, 2024

Related U.S. Application Data
(60) Provisional application No. 63/362,288, filed on Mar. 31, 2022.

(51) Int. Cl. A61M 25/10; A61B 17/3207

(52) U.S. Cl. CPC A61M 25/104; A61B 17/320725; A61M 2025/105

(57) ABSTRACT

An intravascular medical device (100) includes a neuromodulation catheter (102) configured to be navigated through vasculature of a patient and deliver denervation therapy to tissue at a target treatment site of a vessel. The intravascular medical device also includes an expandable member (112) configured to deliver a therapeutic agent disposed on an outer surface of the expandable member to an inner surface of the vessel when the expandable member is expanded.

### TECHNICAL FIELD

The present technology is related to intravascular medical devices, such as ablative medical devices.

### BACKGROUND

Balloon angioplasty may be used to treat cardiovascular diseases involving abnormal constriction or enlargement of blood vessels, such as cerebrovascular disease, coronary heart disease, and peripheral arterial disease. These abnormal constrictions or enlargements may be caused by underlying tissue morphology near smooth muscle tissue lining the blood vessel wall, such as fatty plaques.

### SUMMARY

The present disclosure describes devices, systems, and methods for delivery of a therapeutic agent to a vessel to modify a structure of the vessel, such as enlarge the vessel. In examples described herein, an intravascular medical device, such as a catheter, is configured to be positioned within a vessel at or proximate a target treatment site. A distal end of the intravascular medical device includes a member configured to deliver the therapeutic agent to a wall of the vessel at the target treatment site. The distal end includes an expandable member with a therapeutic agent disposed on an outer surface of the expandable member, such that the therapeutic agent contacts and deposits onto the wall of the vessel at the target treatment site when the expandable member is expanded.

Once the expandable member has delivered the therapeutic agent to the vessel at the target treatment site, the intravascular medical device is removed from the vessel, leaving the therapeutic agent on or in the wall of the vessel. The therapeutic agent is configured to induce cell death and/or inhibit proliferation of cells by various mechanisms at or proximate the target treatment site over a period of time. For example, the therapeutic agent may induce cell death of a portion of smooth muscle cells over a first period of time and inhibit the growth and/or replacement of the portion of smooth muscle cells and endothelial cells over a second period of time subsequent the first period of time. In the absence of the portion of smooth muscle cells, blood pressure within the vessel may cause the vessel and/or lumen of the vessel at and/or proximate the target treatment site to enlarge and/or dilate. The period of time for which living cells may be ablated or inhibited may be on the order of weeks or months, and the removal and/or absence of the tissues may be less traumatic to the vessel and/or may delay and/or distribute healing over a longer period of time, e.g., relative to a therapy configured to remove smooth muscle cells over a shorter period of time, such as hours or days. The reduced trauma and/or delayed healing may reduce inflammation of the vessel at and/or proximate the target treatment site.

In one example, this disclosure describes an intravascular medical device including a neuromodulation catheter configured to be navigated through vasculature of a patient and deliver denervation therapy to tissue at a target treatment site of a vessel; and an expandable member configured to deliver a therapeutic agent disposed on an outer surface of the expandable member to an inner surface of the vessel when the expandable member is expanded.

In another example, this disclosure describes a method including delivering denervation therapy to a tissue adjacent to a vessel of a patient at a denervation target site; and after delivering the denervation therapy, expanding an expandable member proximate to the tissue to contact a therapeutic agent disposed on an outer surface of the expandable member to an inner surface of the vessel and deliver the therapeutic agent to the vessel.

In another example, this disclosure describes an intravascular medical device including an elongated member configured to be navigated through vasculature of a patient to a target treatment site in a vessel of the patient; one or more therapeutic elements positioned at a distal end of the elongated member, wherein the one or more therapeutic elements are configured to deliver neuromodulation therapy to the target treatment site; and an expandable member configured to be delivered to the target treatment site via the elongated member, wherein the expandable member is configured to deliver a therapeutic agent dispose on an outer surface of the expandable member to an inner surface of the vessel when expanded.

In another example, this disclosure describes a method including expanding an expandable member proximate to tissue adjacent to a vessel of a patient at a target treatment site, wherein expanding the expandable member causes an outer surface of the expandable member to contact a therapeutic agent disposed on an outer surface of the expandable member to an inner surface of the vessel at the target treatment site and to deliver the therapeutic agent to the vessel, wherein the therapeutic agent is configured to remove a portion of smooth muscle cells of the vessel over a first period of time. The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout and wherein:

FIG. 1 is a partially schematic illustration of a vascular cold therapy system that includes an example intravascular medical device, in accordance with some examples of the present disclosure.

FIG. 2 illustrates a technique for accessing a renal artery and modulating renal nerves with the system of FIG. 1 in accordance with some examples of the present disclosure.

FIG. 3 is a conceptual illustration of an example sympathetic nervous system (SNS) and how the brain communicates with the body via the SNS.

FIG. 4 is an enlarged anatomic view of nerves innervating a left kidney to form the renal plexus surrounding the left renal artery.

FIGS. 5 and 6 are anatomic and conceptual views, respectively, of a human body depicting neural efferent and afferent communication between the brain and kidneys.

FIGS. 7 and 8 are anatomic views of the arterial vasculature and venous vasculature, respectively, of a human.

FIG. 9 is an expanded side view conceptual illustration of a distal portion of an example intravascular medical device positioned in a vessel in a delivery configuration.

FIG. 10 is an expanded side view conceptual illustration of the distal portion of the example intravascular medical device of FIG. 9 positioned in the vessel in a deployed configuration.

FIG. 11 is an expanded side view conceptual illustration of the distal portion of the example intravascular medical device of FIG. 9 positioned in the vessel in an expanded configuration.

FIG. 12 is an expanded side view conceptual illustration of a distal portion of an example intravascular medical device positioned in a vessel in a delivery configuration after delivering a therapeutic agent to the vessel.

FIG. 13 is a cross-sectional illustration of a vessel with a therapeutic agent.

FIG. 14 is a flowchart of an example method for delivering a therapeutic agent to a wall of a vessel, in accordance with some examples of the present disclosure.

FIG. 15 is an example timing graph illustrating a biological response to the therapeutic agent over a period of time.

### DETAILED DESCRIPTION

The present technology is directed to devices, systems, and methods for treatment of a vessel.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Conditions such as arrhythmias, hypertension, states of volume overload (e.g., heart failure), and progressive renal disease due to excessive activation of the renal sympathetic nervous system (SNS), may be mitigated by modulating the activity of overactive nerves (neuromodulating), for example, denervating or reducing the activity of the overactive nerves. Sympathetic nerves of the kidneys terminate in the renal blood vessels, the juxtaglomerular apparatus, and the renal tubules, among other structures. The overactive nerves may be chemically, thermally, mechanically, and/or electrically denervated by ablating sympathetic nerve tissue in or near renal blood vessels. Therapy may be delivered to the sympathetic tissue via navigating a catheter including, including therapeutic elements such as needles and/or electrodes, within the vasculature of the patient. The therapeutic elements deliver therapy to the tissues, such as directly to the wall of the vessel or by extending elements into or through the wall of the vessel. For example, in the case of chemical renal denervation, one or more needles may radially extend from the catheter to puncture a vessel wall to deliver the chemical and/or a cold therapy fluid via a needle lumen to ablate tissue at a target treatment site.

Ablation of nervous tissue may involve varying degrees of interaction with tissues of the wall of the vessel. In some instances, the wall of the vessel may remain relatively intact. For example, ablation using chemical therapy may result in targeted ablation that does not substantially ablate tissues of the wall of the vessel. In some instances, various tissues of the wall of the vessel may be modified. For example, ablation using heat or cold therapy may ablate living cells of the wall of the vessel, such as smooth muscle cells of the tunica adventitia or endothelial cells of the tunica intima. Post ablation, the vessel may undergo several phases of rebuilding, which may include inflammation and other vessel responses, polymerization of proteins denatured during ablation into collagen, and regrowth of living cells, such as endothelial cells, to reform the vessel. However, healing of the vessel may result in nonconcentric and/or constricted renal arteries post ablation/treatment. For example, a vessel in which smooth muscle cells have been ablated in only a portion of the wall of the vessel may have a nonconcentric shape. In some instances, a size of the vessel may continue to influence activity of the renal system. For example, a relatively small renal artery may deliver a reduced blood flow to a kidney. The renal system may induce various physiological changes to increase blood pressure and deliver a greater amount of blood to the kidney.

In accordance with examples of the current disclosure, an intravascular medical device is configured to deliver a therapeutic agent to remove and/or inhibit the growth of smooth muscle cells of the vessel at and/or proximate to a target treatment site over a period of time. By establishing a relatively uniform expanded lumen within the vessel, healing may occur in an improved manner and may reduce a risk arterial stenosis. In the absence of the smooth muscle cells, blood pressure within the vessel may cause the vessel and/or lumen of the vessel at and/or proximate the target treatment site to enlarge and/or dilate, e.g., limited by the extent of the extra cellular matrix of the vessel. The period of time may be on the order or weeks or months. In some examples, the therapeutic agent may be configured to allow the vessel to dilate, enlarge, and/or increase in size over at least one of the periods of time without an expandable member, balloon, stent, or the like, causing and/or maintaining the dilation and/or enlargement of the vessel. For example, the expandable member may be configured to deliver the therapeutic agent to an inner surface of a vessel wall via contacting the vessel wall with therapeutic agent and without substantially increasing the diameter and/or circumference of the lumen, e.g., substantially without pushing on the inner surface of the vessel wall and/or without creating any injury to the vessel or vessel wall, such as micro-injuries or micro-tears in the vessel or vessel wall and/or vessel wall dissections. For example, the expandable member may be expanded with a limited pressure so as to contact the inner surface of the vessel wall with the therapeutic agent without substantially exerting a force or pushing on the inner surface of the vessel wall. In some examples, an amount that the expandable member increases the perimeter length of the inner surface of the vessel wall is less than 20%, or less than 10%, or less than 5%, or less than 1% beyond its perimeter length prior to any inflation of the balloon 312.

Setting the vessel in a relatively uninform shape and expanded size may create a blueprint for healing phases that may improve post treatments, reduce vessel irregularities, and create an environment where thrombus is reduced and healing response is improved. The removal/absence of the portion of smooth muscles cells over the period of time may be less traumatic to the vessel and may delay and/or distribute healing over a longer period of time, e.g., relative to a therapy configured to remove smooth muscle cells over a shorter period of time, such as hours or days. The reduced trauma and/or delayed healing may reduce inflammation of the vessel at and/or proximate the target treatment site. Although examples herein describe systems, devices, and techniques in the context of post denervation treatment (such as renal denervation), the systems, methods, and techniques of the current disclosure are not so limited.

In accordance with examples of the current disclosure, the therapeutic agent may be delivered to a vessel using an intravascular medical device that both delivers an acute therapy for denervating the nervous tissue around the vessel and chronic therapy for inducing cell death and/or inhibiting cells of the wall of the vessel. In such examples, an intravascular medical device may include a neuromodulation catheter configured to be navigated through vasculature of a patient and deliver denervation therapy to tissue in a denervation target area of a vessel. The intravascular medical device further includes an expandable member configured to contact the outer surface of the expandable member to an inner surface of the renal vessel when the expandable member is expanded, e.g., the expandable member may be a balloon. A therapeutic agent is disposed on an outer surface of the expandable member and delivered to the inner surface of the vessel when the expandable member is expanded to contact the vessel. The therapeutic agent is configured to remove a portion of smooth muscle cells of the vessel over a period of time. In response to regular blood pressure within the vessel, the wall of the vessel may expand to an increased cross-sectional area to the extent permitted by the extracellular matrix of the wall of the vessel. Once the therapeutic agent has reduced or been consumed, the endothelial and smooth muscle cells may grow back to the increased cross-sectional area. The resulting vessel may have increased flow with reduced inflammation.

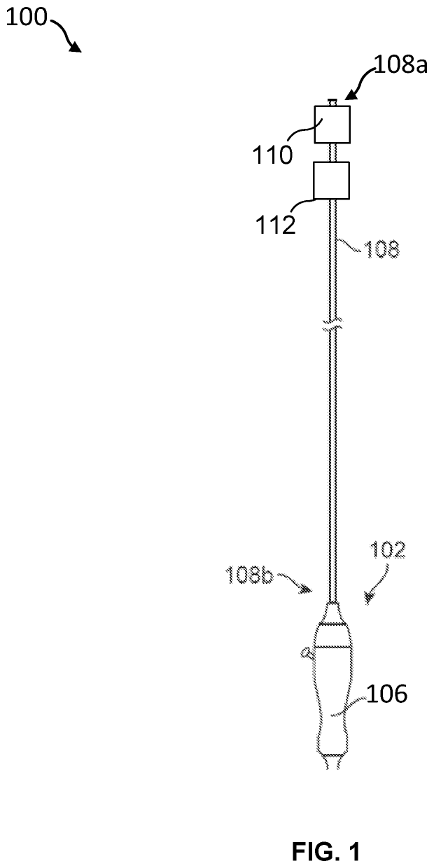

FIG. 1 is a schematic illustration of an intravascular medical device 100 configured in accordance with some examples of the present technology. Intravascular medical device 100 includes a catheter 102. In the example of FIG. 1, catheter 102 includes one or more therapeutic elements 110 and an expandable member 112. Although described in FIG. 1 as including a single catheter, in some examples, intravascular medical device 100 may include a first catheter including therapeutic elements 110 and a second catheter 102 including an expandable member 112. In other examples, catheter 102 may include both therapeutic elements 110 during delivery of a first therapy (e.g., neuromodulation therapy) and expandable member 112 during delivery of a subsequent second therapy. In the example shown, catheter 102 includes both therapeutic elements 110 and expandable member 112, e.g., during delivery of both the first therapy via therapeutic elements 110 and delivery of the second therapy via expandable member 112.

Catheter 102 includes a handle 106 and an elongated member, e.g., elongated member 108 attached to the handle 106. Elongated member 108 may include a distal portion 108a and a proximal portion 108b. Elongated member 108 may have any suitable outer diameter, and the diameter can be constant along the length of elongated member 108 or may vary along the length of elongated member 108. In some examples, elongated member 108 can be 2, 3, 4, 5, 6, or 7 French or another suitable size.

Distal portion 108a of elongated member 108 is configured to be moved within an anatomical lumen of a human patient to locate therapeutic elements 110 and expandable member 112 at a target treatment/denervation site within, or otherwise proximate to, the anatomical lumen. For example, elongated member 108 may be configured to position therapeutic elements 110 within a blood vessel, a ureter, a duct, an airway, or another naturally occurring lumen within the human body. The following description focuses on positioning distal portion 108a and therapeutic elements 110 within a blood vessel. A person having ordinary skill in the art will understand that the description and examples described herein are also applicable to positioning distal portion 108a and therapeutic elements 110 within other anatomical lumens. In some examples, elongated member 108 is structurally configured to be relatively flexible, pushable, and relatively kink-and buckle-resistant, so that it may resist buckling when a pushing force is applied to handle 106 and/or proximal portion 108b to advance elongated member 108 distally through vasculature, and so that it may resist kinking when traversing around a tight turn in the vasculature. Elongated member 108 may have any suitable shape, such as a tubular body or a paddle-like shape. Elongated member 108 may be constructed using any suitable materials. In some examples, elongated member 108 may include one or more polymeric materials, for example, polyamide, polyimide, polyether block amide copolymer sold under the trademark PEBAX, polyethylene terephthalate (PET), polypropylene, aliphatic, polycarbonate-based thermoplastic polyurethane, or a polyether ether ketone (PEEK) polymer that provides elongated member 108 with a predetermined flexibility. The polymeric materials may be extruded as one or more solid or hollow tubes to form elongated member 108.

In some examples, a support structure or shape member may be included within or about elongated member 108, for example, being disposed about, within, or between one or more polymeric tubes used to form elongated member 108. The support structure or shape member may be used to impart a predetermined strength, flexibility, shape, or geometric qualities to elongated member 108. The support structure or shape member may be formed using any suitable materials including, for example, metal, alloy, or polymer-based wires used to form coils or braids, a hypotube, shape memory materials, for example, nickel-titanium (nitinol), shape memory polymers, electro-active polymers, or the like. The support structure or shape member may be cut using a laser, electrical discharge machining (EDM), electrochemical grinding (ECG), or other suitable means to achieve a desired finished component length, apertures, and geometry. In some examples, the support structure or shape member may be arranged in a single or dual-layer configuration, and manufactured with a selected tension, compression, torque and pitch direction.

Elongated member 108 may also include one or more radiopaque markers which may help a clinician determine the positioning of elongated member 108, e.g., therapeutic elements 110, expandable member 112, or a distal end of elongated member 108, relative to the target treatment site using ultrasound or other suitable technique. For example, one or more radiopaque markers may be positioned along elongated member 108 such as near a distal end, adjacent to therapeutic elements 110, expandable member 112, or the like.

In some examples, at least a portion of an outer surface of elongated member 108 may include one or more coatings, such as, but not limited to, an anti-thrombogenic coating, which may help reduce the formation of thrombi in vitro, an anti-microbial coating, and/or a lubricious coating. In some examples, the entire working length of elongated member 108 may be coated with the hydrophilic coating. In other examples, only a portion of the working length of elongated member 108 coated with the hydrophilic coating. This may provide a length of elongated member 108 distal to handle 106 with which the clinician may grip elongated member 108, e.g., to rotate elongated member 108 or push elongated member 108 through vasculature. In some examples, the entire working length of elongated member 108 or portions thereof may include a lubricious outer surface, e.g., a lubricious coating. The lubricating coating may be configured to reduce static friction and/or kinetic friction at a surface of elongated member 108 as elongated member 108 is advanced through the vasculature.

Proximal portion 108b of elongated member 108 may be received within handle 106 and can be mechanically connected to handle 106 via an adhesive, welding, or another suitable technique or combination of techniques. Handle 106 may serve as a handle for catheter 102 allowing the clinician to grasp catheter 102 at handle 106 and advance elongated member 108 through vasculature of a patient. In some examples, catheter 102 can include another structure in addition or instead of handle 106. For example, catheter 102 or handle 106 may include one or more luers or other mechanisms (e.g., access ports) for establishing connections between catheter 102 and other devices. Additionally, or alternatively, catheter 102 may include a strain relief body (not shown), which may be a part of handle 106 or may be separate from handle 106 to alleviate potential strain of kinking of elongated member 108 near its proximal end.

In some examples, distal portion 108a of catheter 102 may include one or more therapeutic elements 110 configured to deliver a neuromodulation therapy, e.g., catheter 102 may be a neuromodulation catheter. In some examples, therapeutic elements 110 may be configured to deliver denervation therapy that includes at least one of delivery of cold therapy, delivery of heat therapy, delivery of radiofrequency energy, delivery of ultrasound energy, or delivery of a chemical agent. Therapeutic elements 110 may be positioned around (e.g., distributed around) a circumference of distal portion 108a.

Distal portion 108a may include any number of therapeutic elements 110. For example, distal portion 108a may include one, two, three, four, or more therapeutic elements 110 positioned around a circumference of distal portion 108a at a single longitudinal position. As another example, distal portion 108a may include two, three, four, or more therapeutic elements 110 positioned around a circumference of distal portion 108a at each of multiple longitudinal positions along distal portion 108a. In examples in which distal portion 108a includes therapeutic elements 110 positioned at different longitudinal positions, each longitudinal position may include one or more therapeutic element 110, and each longitudinal position may include the same number of therapeutic elements 110, or one longitudinal position may include a different number of therapeutic elements 110 than one or more other longitudinal positions.

Intravascular medical device 100 includes expandable member 112. Expandable member 112 is mechanically connected to and carried by elongated member 108. Expandable member 112 may be positioned around (e.g., distributed around) a circumference of distal portion 108a. In some examples, such as illustrated in FIG. 1, expandable member 112 is positioned at distal portion 108a of elongated member 108 proximal to therapeutic elements 110; in other examples, expandable member 112 may be positioned elsewhere on elongated member 108. Expandable member 112 is configured to be positioned within a lumen of the vessel and expanded within the lumen of the vessel such that an outer surface of expandable member 112 contacts an inner surface of the vessel at, proximate to, the target treatment site. For example, expandable member 112 may include an expansion structure configured to expand from a radially collapsed delivery configuration to a radially expanded deployed configuration. In some examples, expandable member 112 may be configured to expand beyond an initial diameter of the vessel, such that surfaces of expandable member 112 may dilate and/or expand the vessel and increase a surface area of expandable member 112 in contact the wall of the vessel. Any of a variety of expandable structures may be used for expandable member 112 including, but not limited to, a balloon, a cage, a mesh, a coil, a braid, and the like.

Expandable member 112 includes a therapeutic agent disposed on an outer surface of expandable member 112. The therapeutic agent may be present in a delivery medium, such as a polymer matrix, coating, or other layer, on the surface of expandable member 112. Expandable member 112 is configured to deliver the therapeutic agent to the wall of the vessel. For example, expandable member 112 may expand to deliver a polymer matrix that includes the therapeutic agent to a surface of the wall of the vessel and collapse to leave the polymer matrix on the wall of the vessel. In some examples, expandable member 112 may be a drug coated balloon (DCB) including a drug and/or therapeutic agent.

The therapeutic agent is configured to induce cell death and/or inhibit cellular migration and/or cellular secretion of signaling factors or extracellular matrix proteins and/or inhibit proliferation/growth of cells of the wall of the vessel over a period of time. These functions encompass all cell types including resident smooth muscle cells, adventitial cells, and incoming inflammatory cells. A vessel, such as a renal artery, includes various layers, such as an inner tunica intima, an intermediate tunica media, and an outer tunica adventitia. The tunica intima includes endothelial cells and, in certain arteries, an elastic sheath and/or smooth muscle cells. The endothelial cells may form an interface between the wall of the vessel and blood flowing in the lumen of the vessel. The tunica media includes smooth muscle cells and structural proteins, such as elastin and collagen fibers. The smooth muscle cells may provide rigid support to the wall, as well as an ability to constrict or relax the artery, and thus assist in regulation of blood flow and pressure, and the structural proteins may provide plasticity and elasticity to the wall. The tunica adventitia also includes structural proteins, which may perform a similar function as structural proteins of tunica media.

The therapeutic agent may include one or more drugs, toxins, or other substances that migrate from the delivery medium into tissues of the wall of the vessel and induce cell death of living cells in the wall of the vessel. For example, the therapeutic agent may induce cell death of endothelial cells in the tunica intima and smooth muscle cells in the tunica media. The therapeutic agent may continue to migrate from the delivery medium into tissues of the wall of the vessel and inhibit growth of cells in the wall of the vessel. For example, the therapeutic agent may reduce or prevent proliferation of endothelial cells and smooth muscle cells to permit the vessel to expand in response to blood pressure in the vessel.

The delivery medium may be configured to release the therapeutic agent(s) over a period of time to provide a controlled restructuring of the vessel. The therapeutic agent or a combination of agents may be configured to induce cell death of the cells over a first period of time that is two weeks or more, one month or more, three months or more, six months or more, up to nine months, or any suitable time period. The first period of time may be configured to provide a relatively slow ablation of the smooth muscle cells, which may result in a more uniform vessel shape and/or reduced inflammation of tissues in or near the wall of the vessel. The therapeutic agent may be configured to inhibit proliferation of cells over a second period of time that is two weeks or more, one month or more, three months or more, six months or more, up to nine months, or any suitable time period. The second period of time may be configured such that the vessel may expand and adapt to the reduced recoil no longer provided by the smooth muscle cells prior to regrowth of endothelial cells. In some examples, the first period of time and the second period of time may overlap. In some examples, the first and/or second periods of time over which the therapeutic agent is configured to remove, and/or inhibit the growth of, the smooth muscle cells, and/or the amount of the portion of smooth muscle cells the therapeutic agent is configured to remove and/or inhibit the growth of, may be defined by the amount of therapeutic agent disposed on the outer surface of expandable member 112.

FIG. 15 is an example timing graph illustrating a biological response to the therapeutic agent over a period of time. While illustrated as linear and precisely coordinated, the graph in FIG. 15 is intended as a general illustration of the timing relationship of the therapeutic agent to various biological responses of the wall of the vessel. As shown in FIG. 15, delivery of the therapeutic agent to the wall of the vessel by expansion device 112 initiates migration of the therapeutic agent into the wall of the vessel and ablation of cells of the wall of the vessel (tₖ), including smooth muscle cells and endothelial cells. In some instances, death of the endothelial cells may be relatively fast, as the endothelial cells may be removed by expansion of expansion device 112.

Upon death of the smooth muscle cells, the vessel may have reduced elastic recoil resulting in expansion of the vessel diameter. While illustrated as gradual and relatively linear, this expansion may be relatively quick or leveling off. The therapeutic agent may continue to migrate into to wall of the vessel to inhibit growth of the endothelial cells and smooth muscle cells (tᵢ), thereby enabling the vessel to expand. The therapeutic agent may be configured to release into the wall of the vessel for a sufficient amount of time to allow the vessel to expand to a greater diameter in response to blood pressure of blood in the vessel. In examples in which an ablative procedure (e.g., renal neuromodulation), rather than the therapeutic agent, removes and/or induces cell death of the smooth muscle cells, the therapeutic agent may be configured to inhibit the growth of the smooth muscle cells and endothelial cells without initially inducing cell death of the smooth muscle cells and endothelial cells. In such examples, the structural proteins of the wall of the vessel may also be modified or denatured, resulting in cross-linking of the structural proteins.

Once the therapeutic agent in the delivery medium is exhausted, the smooth muscle cells and endothelial cells may begin to regrow to form an intact wall of the vessel (tₕ). Once the vessel is repaired (tᵣ), the vessel may have a greater diameter and/or more uniform shape than prior to modification of the vessel, which may result in increased blood flow and/or fewer complications that a vessel which has not been enlarged in a controlled manner using a therapeutic agent over a relatively long period of time.

Referring back to FIG. 1, in certain examples, intravascular delivery of the therapeutic elements 110 and/or expandable member 112 includes percutaneously inserting a guidewire (not shown) into a blood vessel of a patient and moving elongated member 108, therapeutic elements 110, and/or expandable member 112 along the guidewire until therapeutic elements 110 reaches a target site (e.g., a renal vessel, such as a renal artery or renal vein). For example, the distal end of elongated member 108 may define a passageway for engaging the guidewire for delivery of therapeutic elements 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide catheter or sheath (not shown), or other guide device.

Once catheter 102 is positioned at the target treatment site, intravascular medical device 100 may be configured to deliver a first therapy to the vessel at the target treatment site via therapeutic elements 110 and a second therapy to the vessel at the target treatment site via expandable member 112. For example, intravascular medical device 100 may be configured to deliver a neuromodulation therapy to the target site via therapeutic elements 110, and then to deliver the therapeutic agent to the vessel at the target treatment site via expanding expandable member 112.

Therapeutic elements 110 can be configured to deliver therapy, such as RF energy, microwave energy, ultrasound energy, a chemical agent, or the like, to provide or facilitate neuromodulation therapy at the target treatment site. By having therapeutic elements 110 located around a circumference of distal portion 108a, neuromodulation catheter 102 may be used to deliver the neuromodulation therapy around a circumference of the blood vessel in which distal portion 108a is positioned. While a circumference of the blood vessel is generally referred to herein, the blood vessel may not be perfectly circular in cross-section and may have any suitable geometry in cross-section.

In some instances, therapeutic elements 110 may replace or supplement initial ablation of cells of the wall of the vessel by the therapeutic agent (e.g., during the first period of time described above). During the course of delivering the neuromodulation therapy, therapeutic elements 110 may be configured to induce cell death of cells of the vessel. For example, certain ablation modalities, such as cold therapy, may be delivered around a circumference of the wall of the vessel. As a result, the therapy may ablate smooth muscle cells around the circumference of the vessel, such that the therapeutic agent may only be configured to inhibit future growth of cells.

In some examples, expandable member 112 may be configured to expand and/or dilate the vessel before, during, or after delivery of neuromodulation therapy and/or the therapeutic agent. For example, expansion and/or dilation of the vessel may increase a surface area of the wall of the vessel exposed to the therapeutic agent, disrupt calcification, scar tissue, or other structures in or on the wall of the vessel, and/or assist in removing endothelial cells from the wall of the vessel. In some examples, intravascular medical device 100 may include a second expandable member configured to dilate the vessel at the target treatment site. For example, intravascular medical device 100 may be configured the expand the second expandable member to dilate the vessel and/or stabilize and/or maintain at least a portion of distal portion 108a substantially stationary relative to the wall of the blood vessel in which distal portion 108a is positioned, e.g., in preparation for delivery of the first therapy via therapeutic elements 110 and/or delivery of the second therapy via expandable member 112.

In some examples, intravascular medical device 100 may be configured to deliver the first and second therapies at the same time or in any order, e.g., the second therapy may be delivered before the first therapy. For example, expandable member 112 may be configured to expand to stabilize and/or maintain at least a portion of distal portion 108a substantially stationary relative to the wall of the blood vessel in which distal portion 108a is positioned, and to deliver the therapeutic agent, in preparation for delivery of the first therapy via therapeutic elements 110. In some examples, intravascular medical device 100 may be configured to deliver the first and second therapies at first and second target treatment sites. For example, intravascular medical device 100 may be configured to deliver the first therapy to a first target treatment site via therapeutic elements 110 and the second therapy at a second target treatment site via expandable member 112. The first and second target treatment sites may be the same or different from each other. In some examples, expandable member 112 may be a part of, integral to, or may be therapeutic elements 110. For example, therapeutic elements 110 may include one or more expandable members of a cryogenic catheter, or one or more expandable members at least partially surrounding an element for delivering energy such as RF energy, microwave energy, ultrasound energy, a chemical agent, or the like, and the one or more expandable member may include the therapeutic agent disposed on its outer surface. In other words, therapeutic elements 110 may also have the functionality of expandable member 112, e.g., including the therapeutic agent disposed on an outer surface of an expandable member of therapeutic elements 110. In some examples, such a combined therapeutic elements 110/expandable member 112 may have a faster procedure time.

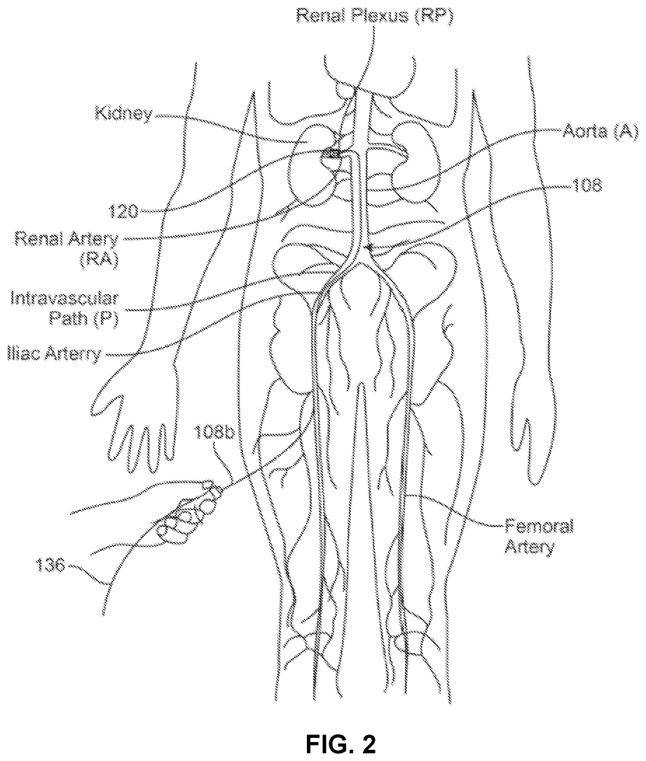

FIG. 2 (with additional reference to FIG. 1) illustrates gaining access to renal nerves of an example patient in accordance with some examples of the present technology. Neuromodulation catheter 102 provides access to the renal plexus RP through an intravascular path P, such as a percutaneous access site in the femoral (illustrated), brachial, radial, or axillary artery to a targeted treatment site within a respective renal artery RA. By manipulating proximal portion 108b of elongated shaft 108 from outside the intravascular path P, a clinician may advance at least distal portion 108a of elongated shaft 108 through the sometimes tortuous intravascular path P and remotely manipulate distal portion 108a (FIG. 1) of elongated shaft 108.

In the example illustrated in FIG. 2, therapeutic elements (e.g., therapeutic elements 110; not shown) are delivered intravascularly to the treatment site using a guidewire 136 in an OTW technique. A neuromodulation assembly 120 may define a passageway for receiving guidewire 136 for delivery of the neuromodulation catheter 102 using either an OTW or a RX technique. At the treatment site, guidewire 136 can be at least partially withdrawn or removed, and therapeutic elements 110 can transform or otherwise be moved to a deployed arrangement for delivering a neuromodulation therapy. In other examples, therapeutic elements 110 may be delivered to the treatment site within a different guide device, such as a guide sheath (not shown), with or without using guidewire 136. In examples in which the system includes a guide sheath, when therapeutic elements 110 are at the target site, the guide sheath may be at least partially withdrawn or retracted and therapeutic elements 110 may be transformed into the deployed arrangement. For example, at least a portion of therapeutic elements 110 may be self-expandable such that they expand to the deployed arrangement upon being released from the guide sheath. In still other examples, elongated shaft 108 may be steerable itself such that therapeutic elements 110 may be delivered to the target treatment site without the aid of guidewire 136 and/or a guide sheath.

An imaging device may enable image guidance, e.g., computed tomography (CT), fluoroscopy, intravascular ultrasound (IVUS), optical coherence tomography (OCT), intracardiac echocardiography (ICE), or another suitable guidance modality, or combinations thereof, to be used to aid the clinician's positioning and manipulation of distal portion 108a and therapeutic elements 110. For example, a fluoroscopy system (e.g., including a flat-panel detector, x-ray, or c-arm) can be rotated to accurately visualize and identify the target treatment site. In other examples, the target treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering therapeutic elements 110. Further, in some examples, image guidance components (e.g., IVUS, OCT) may be integrated with neuromodulation catheter 102 and/or run in parallel with neuromodulation catheter 102 to provide image guidance during positioning of therapeutic elements 110. For example, image guidance components (e.g., IVUS or OCT) can be coupled to therapeutic elements 110 to provide three-dimensional images of the vasculature proximate the target site to facilitate positioning or deploying therapeutic elements 110 within the target renal blood vessel.

As described above, delivery of a therapeutic agent to assist in restructuring of a vessel may be particularly useful when performed in conjunction with renal neuromodulation. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally-induced, chemically-induced, or induced in another suitable manner or combination of manners at one or more suitable target treatment sites during a treatment procedure, such as described with respect to therapeutic elements 110 of FIG. 1. The target treatment site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the tunica adventitia of the renal artery.

Controlled modification (e.g., enlargement or uniformization) of the renal artery may be used to further treat clinical conditions associated with renal neuromodulation and/or biological responses. For example, the renal system may control blood pressure based on blood flow to a kidney from the corresponding renal artery. To reduce blood pressure, the therapeutic agent may be delivered to the renal artery to induce cell death of the smooth muscle cells and permit enlargement of the renal artery, thereby increasing flow to the kidneys. In some instances, inducing cell death of the smooth muscle cells may occur during the course of renal neuromodulation. The therapeutic agent may be delivered to the renal artery to induce cell death of the smooth muscle cells more uniformly and/or permit the vessel to expand prior to repair of the vessel.

The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of treatment devices and associated methods for achieving renal neuromodulation via intravascular access and impose specific design requirements for such devices. Specific design requirements may include accessing the renal artery, positioning therapeutic elements 110 within the renal artery and relative to other physiological structures (such as an accessory renal artery), delivering the chemical agent to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

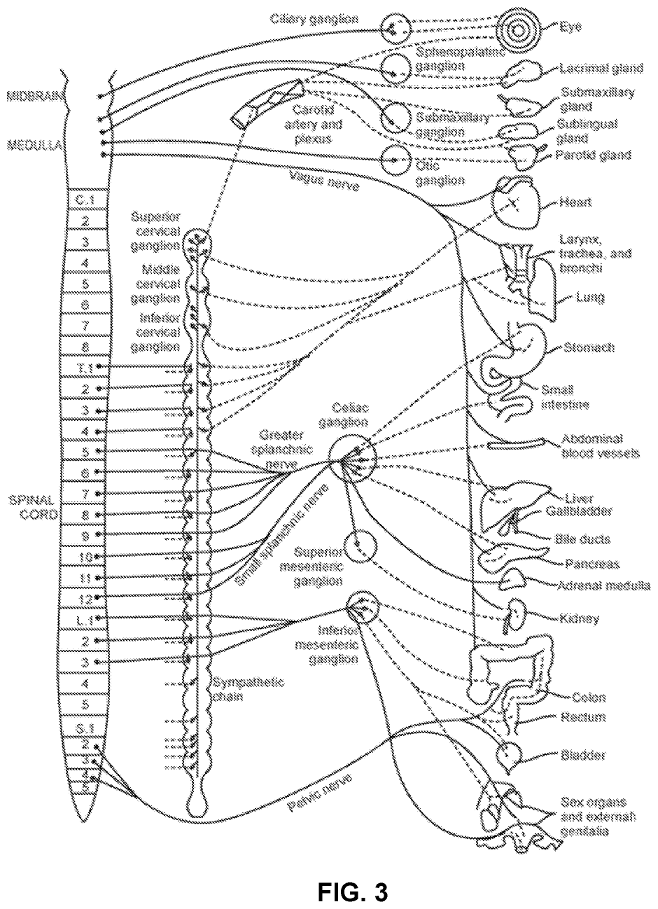

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

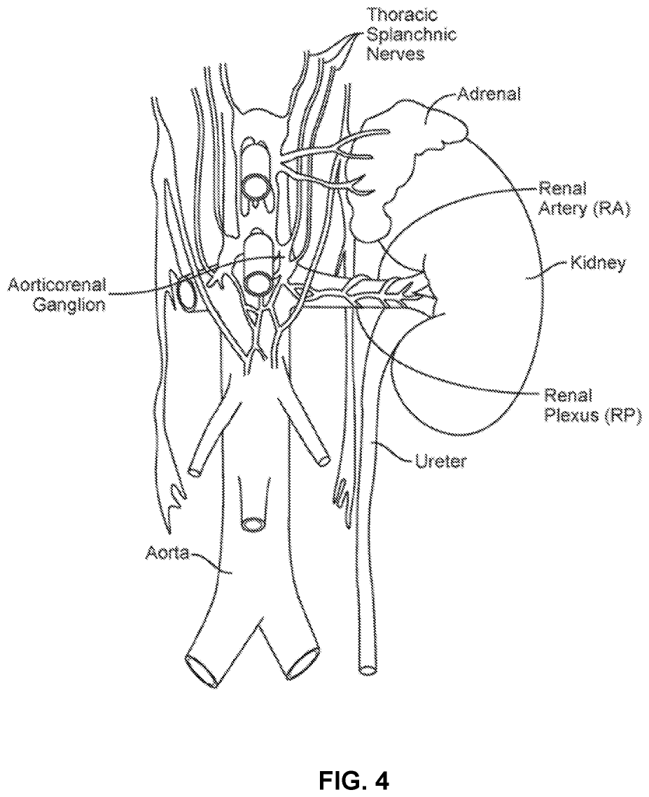

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease in some patients are characterized by heightened sympathetic nervous activation. In patients with end stage renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects and others.

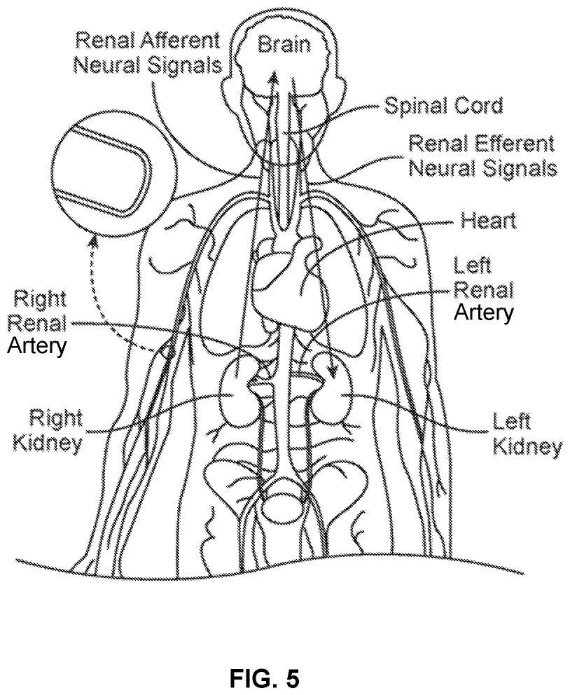

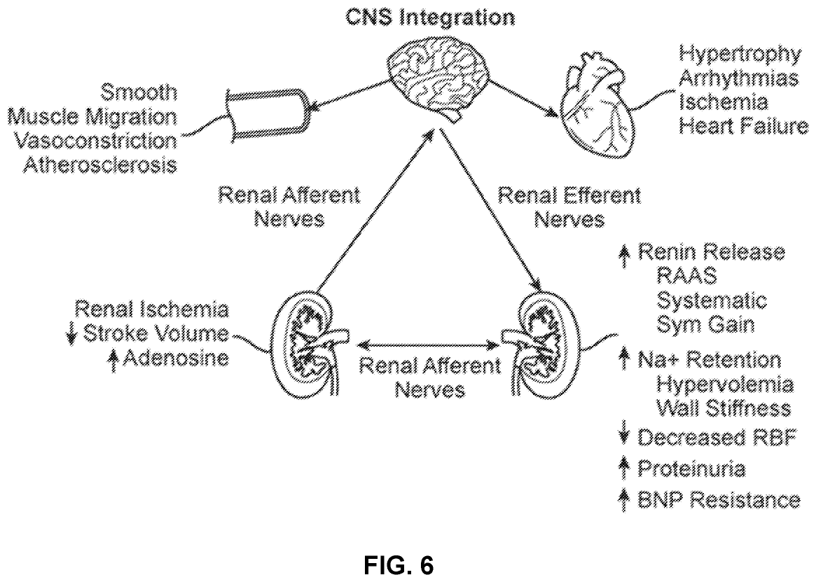

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

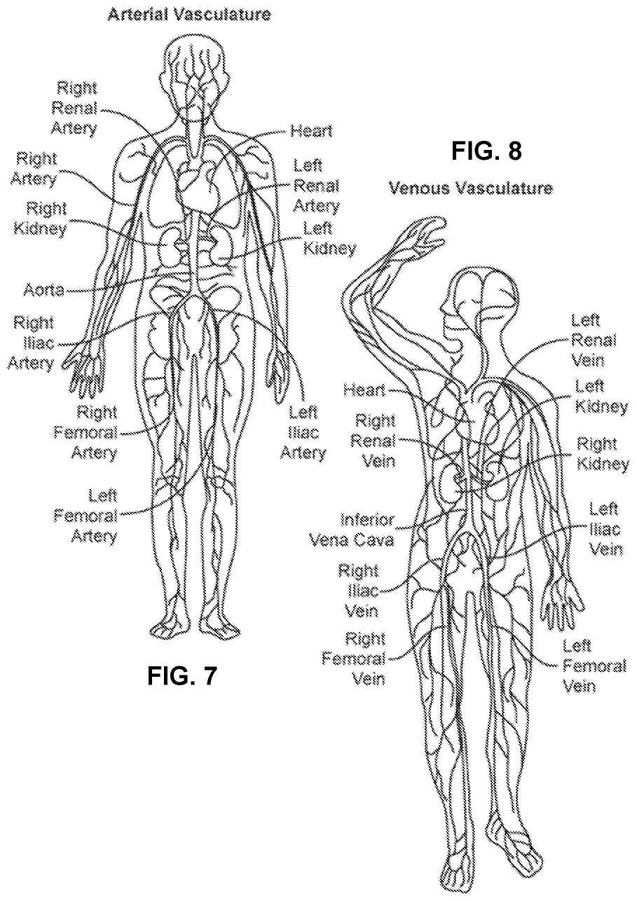

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries. As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels. The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique. Other access sites can also be used to access the arterial system.

The neuromodulatory apparatus may also be configured to allow for adjustable positioning and repositioning of the therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that therapeutic elements 110 and/or expandable member 112 may intimately contact the vessel wall and/or extend at least partially through the vessel wall. Renal artery vessel diameter, D_{RA}, typically is in a range of about 2-10 millimeters (mm), with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the renal artery) to avoid non-target tissue and anatomical structures such as anatomical structures of the digestive system or psoas muscle.

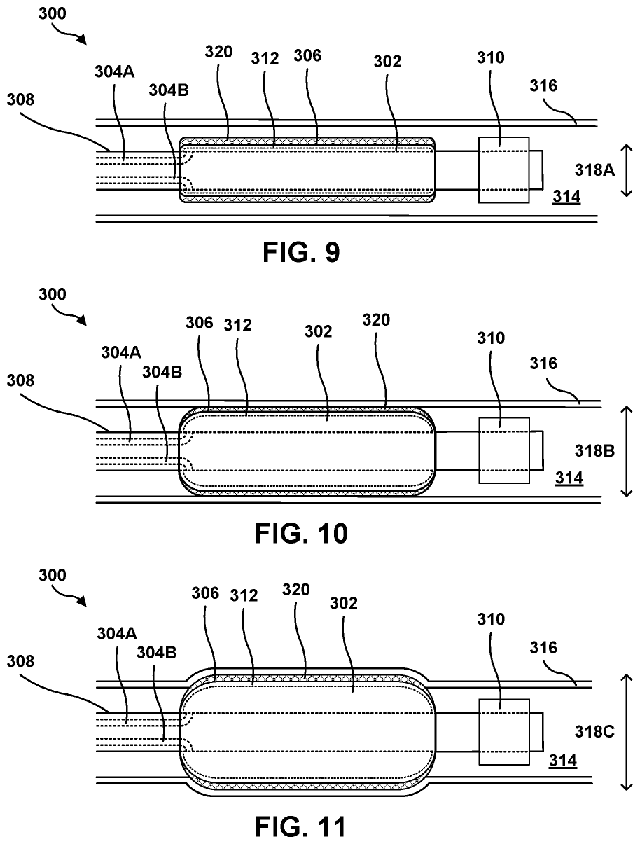

FIG. 9 is an expanded side view conceptual illustration of a distal portion of an intravascular medical device 300 positioned in a lumen 314 of a vessel 316 in a delivery configuration. In some examples, vessel 316 may be a renal vessel. Intravascular medical device 300 may be substantially similar to intravascular medical device 100 described above at FIG. 1. Intravascular medical device 300 includes a balloon 312 having an outer surface 306 and defining a cavity 302. Therapeutic agent 320 is disposed on outer surface 306. When intravascular medical device 300 is in a delivery configuration, such as illustrated in FIG. 9, balloon 312 may be in a deflated configuration in which cavity 302 is empty or nearly empty, such that intravascular medical device 300 may be navigated through vasculature and positioned within lumen 314 of vessel 316 proximate to a treatment site. For example, balloon 312 may have a diameter 318A that corresponds to or is slightly larger than a diameter of an elongated body 308 of intravascular medical device 300. Balloon 312 and therapeutic agent 320 may be substantially similar to expandable member 112 and the therapeutic agent described above at FIG. 1.

Intravascular medical device 300 includes therapeutic elements 310, which may be substantially similar to therapeutic elements 110 described above. In the example shown, therapeutic elements 310 are at a different axial position from balloon 312. In some examples, intravascular medical device 300 may be configured to deliver a first therapy (e.g., denervation or a neuromodulation therapy) via therapeutic elements at a target treatment site, and then intravascular medical device 300 may be configured to move balloon 312 to the target treatment site and deliver a second therapy, e.g., therapeutic agent 320, to vessel 316 at the target treatment site. In other examples, intravascular medical device 300 may be configured to deliver first and second therapies in any order or at the same time, and to the same or different target therapy sites.

Intravascular medical device 300 includes elongated member 308, such as elongated member 108 of FIG. 1. Elongated member 308 includes one or more fluid channels 304A and 304B (individually and collectively “fluid channel 304” and “fluid channel 304,” respectively). Each fluid channel 304 is configured to discharge a fluid into and/or discharge a fluid from cavity 302 of balloon 312. In the example of FIG. 9, elongated member 308 includes a fluid inlet channel 304A configured to discharge a fluid into cavity 302 and a fluid outlet channel 304B configured to discharge the fluid from cavity 302; however, in other examples, elongated member 308 may include a single channel 304, additional channels 304, or channels 304 configured to discharge the fluid both to and from cavity 302. Fluid inlet channel 304A and fluid outlet channel 304B may each be in fluid communication with a fluid source through a port on a hub, such as handle 106 of FIG. 1.

FIG. 10 is an expanded side view conceptual illustration of a distal portion of example intravascular medical device 300 of FIG. 9 positioned in lumen 314 of vessel 316 in a first deployed configuration. Balloon 312 is configured to expand from the deflated configuration in the delivery configuration of intravascular medical device 300 to an inflated configuration in the first deployed configuration of intravascular medical device 300 in response to a pressure differential between an internal pressure within cavity 302 and an external pressure at outer surface 306, such that at least a portion of outer surface 306 of balloon 312 contacts an inner surface of vessel 316. For example, balloon 312 may be made of a semi-compliant, flexible, and/or expandable material. The internal pressure within cavity 302 may be sufficient such that at least a portion of outer surface 306 contacts a target treatment site of vessel 316. In the example shown, therapeutic agent 320 is disposed on outer surface 306, and a portion of therapeutic agent 320 contact the inner surface of vessel 316, e.g., rather than outer surface 306. In some examples, both a therapeutic agent 320 and a portion of outer surface 306 contact the inner surface of vessel 316.

To increase an internal pressure within cavity 302 to expand balloon 312, cavity 302 is configured to receive and contain a pressurized fluid. In some examples, balloon 312 is configured to expand in response to cavity 302 being filled with the pressurized liquid. For example, balloon 312 may be configured to receive a pressurized liquid through fluid inlet channel 304A that fills cavity 302 and expands to a particular pressure of the pressurized liquid. In some examples, balloon 312 is configured to expand in response to cavity 302 being at least partially filled with a pressurized gas. As one example, balloon 312 may be configured to receive a pressurized liquid through fluid channel 304A and expand in response to the pressurized liquid expanding to a lower pressure gas in cavity 302. As another example, balloon 312 may be configured to receive a first reactant and expand as an endothermic reaction between the first reactant and a second reactant produces a gaseous product. As a result of expansion of balloon 312, therapeutic agent 320 and/or a portion of outer surface 306 of balloon 312 may contact an inner surface of a wall of vessel 316. For example, balloon 312 may have a first inflated diameter 318B greater than diameter 318A of FIG. 9 that corresponds to an inner diameter of vessel 316.

Therapeutic agent 320 may be present in a delivery medium. The delivery medium may be configured to release therapeutic agent 320 so that therapeutic agent 320 may migrate into the wall of vessel 316. The delivery medium may control the release of therapeutic agent 320 using a variety of controlled release and/or exposure mechanisms. The delivery medium may be configured to release therapeutic agent 320 over a period of time according to a release rate of therapeutic agent 320 from the delivery medium, a loading of the therapeutic agent in the delivery medium, or the like. The release rate of therapeutic agent 320 may be related to a rate of migration or diffusion of therapeutic agent 320 through tissues of vessel 316, a rate of degradation of a polymer matrix of the delivery medium, or a concentration of therapeutic agent 320 in the delivery medium. In one example, the delivery medium includes a polymer matrix that releases therapeutic agent 320 at a particular release rate corresponding to dissolution of the polymer matrix. The polymer matrix may exhibit tunable physicochemical properties such as permeability, molecular diffusivity, and degradation rate, to release therapeutic agent 320 at a particular release rate that corresponds to an amount of time over which induced cell death of the cells may occur and/or growth the cells may be inhibited. In another example, the delivery medium includes therapeutic agent 320 encapsulated in bioabsorbable polymer shells and dispersed in a polymer matrix, such as a hydrocolloid polymer matrix. The loading of therapeutic agent 320 in the delivery medium may be an amount of therapeutic agent 320 by weight, volume, relative to an amount of the delivery medium, a concentration of therapeutic agent 320 within delivery medium, or the like. The loading of therapeutic agent 320 in the delivery medium may correspond to and/or determine the periods of time over which any or all of therapeutic agent 320 releases (e.g., time tₖ to tₕ of FIG. 15), induced cell death of smooth muscle cells and/or endothelial cells occurs, smooth muscle cells and/or endothelial cells are ablated and/or inhibited from growing (e.g., time tₖ to tᵢ and/or time e.g., time tᵢ to tₕ of FIG. 15), or any suitable period of time to enable the vessel to expand with a relatively reduced amount of inflammation. In these various ways, the delivery medium may maintain a sufficient release of therapeutic agent 320 for a long period of time to inhibit cell growth.

Therapeutic agent 320 and/or the amount of therapeutic agent 320 disposed on outer surface 306 (e.g., in the delivery medium) may be configured to remove a portion of smooth muscle cells of vessel 316 over a first period of time, e.g., two weeks or more, one month or more, three months or more, six months or more, up to nine months, or any suitable time period. Additionally or alternatively, the therapeutic agent 320 and/or the amount of therapeutic agent 320 disposed on outer surface 306 may be configured to inhibit the growth of a portion of smooth muscle cells of vessel 316 over a second period of time, e.g., two weeks or more, one month or more, three months or more, six months or more, up to nine months, or any suitable time period. In some examples, the first period of time and the second period of time may overlap. In some examples, the first and/or second periods of time over which the therapeutic agent is configured to remove, and/or inhibit the growth of, the smooth muscle cells, and/or the amount of the portion of smooth muscle cells therapeutic agent 320 is configured to remove and/or inhibit the growth of, may be defined by the amount of therapeutic agent disposed on outer surface 306.

Therapeutic agent 320 may be configured to remove a portion of the smooth muscle cells of vessel 320 sufficient to allow lumen 314 of the vessel to dilate via blood pressure over at least one of the first period of time or the second period of time. For example, therapeutic agent 320 may be configured to allow lumen 314 of the vessel to dilate, enlarge, and/or increase in size over at least one of the first period of time or the second period of time without an expandable member, balloon, stent, or the like, causing and/or maintaining the dilation and/or enlargement of lumen 314. In some examples, the amount of therapeutic agent 320 is configured to allow vessel 316 to heal with both dilated lumen 314 and with a reduced amount of inflammation over the second time period. In some examples, therapeutic agent 320 may be a chemotherapeutic agent, chemical agent, autoinflammatory agent, neurotoxin or general toxin, or any suitable drug and/or therapeutic agent proven to modify cell behavior as described in (0041) cells. Classes of suitable drugs may include but not limited to chemotherapeutic agents, chemical agents, autoinflammatory agents, neurotoxins and general toxins.

In some examples, intravascular medical device 300 may be configured to transfer at least a portion of therapeutic agent 320 from outer surface 306 to an inner surface of vessel 316. For example, outer surface 306 may be a treated surface configured to have a lower adhesion to therapeutic agent 320 than between the inner surface of vessel 316 and therapeutic agent 320, such that when balloon 312 expands to contact therapeutic agent 320 to vessel 316, at least a portion of therapeutic agent 320 adheres and/or attaches to vessel 316 and detaches from outer surface 306, e.g., upon deflation of balloon 312. In other words, intravascular medical device 300 may be configured to transfer at least a portion of therapeutic agent 320 from outer surface 306, and therapeutic agent 320 may be configured to coat a portion of the inner surface of vessel 316. In some examples, therapeutic agent 320 may be configured to be absorbed by tissue of vessel 316. In some examples, balloon 312 may be configured to expand and apply a pressure to therapeutic agent 320 against the inner surface of vessel 316, and the amount of pressure and the amount of time balloon 312 exerts one or more amounts of pressure, may define an amount of therapeutic agent 320 coated onto, and/or absorbed by, vessel 316.

In the example of FIG. 10, balloon 312 is expanded to an internal diameter of vessel 316. However, in some examples, balloon 312 may optionally be expanded beyond an initial diameter of vessel 316, however, without creating any injury to the vessel or vessel wall, such as micro-injuries or micro-tears in the vessel or vessel wall or vessel wall dissections. For example, the balloon 312 may be inflated, e.g., with limited pressure, to contact the vessel or vessel wall without substantially increasing the diameter and/or circumference of the lumen. In some examples, an amount that the balloon 312 increases the perimeter length of the inner surface of the vessel wall is less than 20%, or less than 10%, or less than 5%, or less than 1% beyond its perimeter length prior to any inflation of the balloon 312. FIG. 11 is an expanded side view conceptual illustration of a distal portion of an example intravascular medical device 300 positioned in an vessel 316 in a second deployed configuration. In the second deployed configuration, an internal pressure of cavity 302 is sufficiently high to expand vessel 316 to a second inflated diameter 318C that is greater than first inflated diameter 318B, but without creating any injury to the vessel or vessel wall, such as micro-injuries or micro-tears in the vessel or vessel wall. In some examples, inflation to the second inflated diameter 318C increases the perimeter length of the inner surface of the vessel wall is less than 20%, or less than 10%, or less than 5%, or less than 1% beyond its perimeter length prior to any inflation of the balloon 312.

In some examples, balloon 312 may include a cutting apparatus. For example, balloon 312 may include one or more blades (not shown). The one or more blades maybe configured to cut one or more portions of vessel 316 to a first depth from the inner surface of vessel 316. Balloon 312 may be configured to deliver at least a portion of the amount of therapeutic agent 320 to tissue adjacent to, or within, the cut portion(s) of vessel 316.

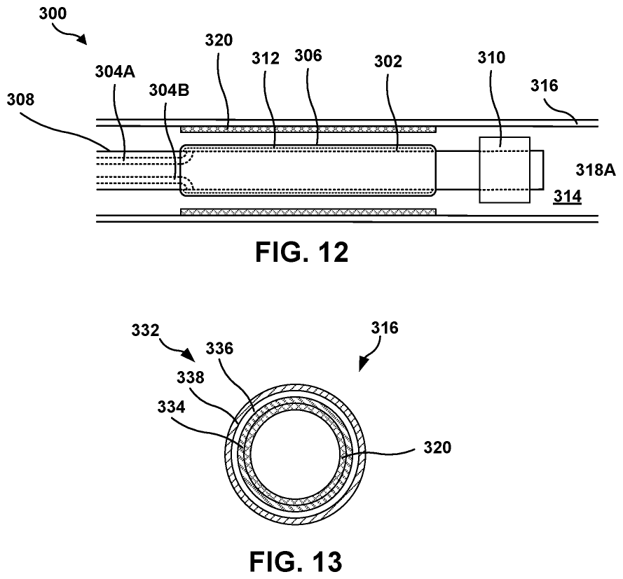

FIG. 12 is an expanded side view conceptual illustration of a distal portion of an example intravascular medical device positioned in a vessel in a delivery configuration after delivering therapeutic agent 320 to vessel 316. Therapeutic agent 320 may remain on an inner surface of vessel 316, such that therapeutic agent 320 may migrate into tissues of vessel 316. FIG. 13 is a cross-sectional illustration of a vessel 316 with therapeutic agent 320. Vessel 316 includes a wall 332 that includes various layers, including an inner tunica intima 334, an intermediate tunica media 336, and an outer tunica adventitia 338. Therapeutic agent 320 may migrate into the various layers to induce cell death of cells of wall 332, such as endothelial cells of tunica intima 334 and smooth muscle cells of tunica adventitia 338.

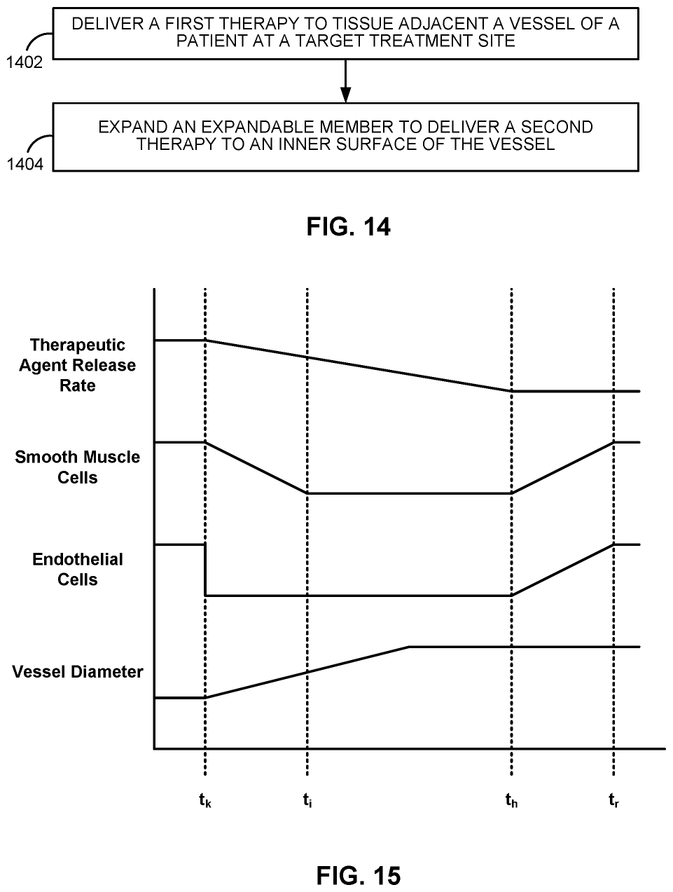

FIG. 14 is a flowchart of an example method for delivering a therapeutic agent to a wall of a vessel, in accordance with some examples of the present disclosure. FIG. 14 will be described with respect to intravascular medical device 100 of FIG. 1 and/or intravascular medical device 300 of FIGS. 9-12, and vessel 316 of FIGS. 9-13, but applies to other examples systems, devices, and target hollow anatomical structures of a patient.

The method of FIG. 14 includes delivering a first therapy to tissue adjacent to a vessel of a patient at a target treatment site (1402). For example, a clinician may manipulate handle 106 and advance distal end of elongated body 308 to a target treatment site of a wall of a vessel 316 of the patient, such that therapeutic elements 310 are positioned proximate the target treatment site. With the aid a medical imaging device, the clinician may confirm a position of therapeutic elements 310 in vessel 316. The wall of vessel 316 includes various living cells, such as endothelial cells and smooth muscle cells, and one or more structural proteins, such as elastin and collagen. The clinician may then manipulate handle 106 and/or a system configured to cause therapeutic elements 310 to deliver the first therapy to the tissue. In some examples, the first therapy may be denervation therapy. In some examples, vessel 316 may be a renal artery and the first therapy may be renal denervation therapy such as delivery of cold therapy, delivery of heat therapy, delivery of radiofrequency energy, delivery of ultrasound energy, delivery of a chemical agent, or the like. Once the first therapy has been delivered to the target treatment site, the clinician may reposition catheter 102 so that expandable member 112, such as balloon 312, is positioned adjacent the target treatment site.

The method of FIG. 14 includes expanding an expandable member to deliver a second therapy to an inner surface of the vessel (1404). For example, subsequent to delivering the first therapy at (1402), the clinician may manipulate handle 106 and/or a system to cause balloon 312 to expand proximate the tissue (e.g., the tissue receiving the first therapy) to contact therapeutic agent 320 disposed on outer surface 306 to an inner surface of vessel 316 and deliver therapeutic agent 320 to vessel 316.

In some examples, the clinician may cause balloon 316 to expand to dilate lumen 314 and/or the walls of vessel 316. In some examples, balloon 316 includes one or more blades and/or cutting devices, and the clinician may cause intravascular medical device 300 to extend the one or more blades to cut a portion of the vessel to a first depth from the inner surface of the vessel, e.g., via expanding of balloon 316. Balloon 316 may then deliver at least a portion of the amount of therapeutic agent 320 to vessel tissue, e.g., adjacent to the cut portion of the vessel and/or within the cut portion of the vessel.

The therapy delivery techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry. The term “processor,” “processing circuitry,” “controller,” or “control circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the intravascular medical device were described as being part of a single device, in other examples, these features can be included on one or more separate devices that can be positioned adjacent to and/or used in tandem with the intravascular medical device to perform similar functions to those described herein.

Clause 1: An intravascular medical device comprising: a neuromodulation catheter configured to be navigated through vasculature of a patient and deliver denervation therapy to tissue at a target treatment site of a vessel; and an expandable member configured to deliver a therapeutic agent disposed on an outer surface of the expandable member to an inner surface of the vessel when the expandable member is expanded.

Clause 2: The intravascular medical device of clause 1, wherein the therapeutic agent is disposed in a delivery medium configured to: adhere to a wall of the vessel; and release the therapeutic agent over a period of time according to at least one of a release rate of the therapeutic agent from the delivery medium or a loading of the therapeutic agent in the delivery medium.

Clause 3: The intravascular medical device of clause 1 or 2, wherein the therapeutic agent is configured to remove a portion of smooth muscle cells of the vessel over a first period of time.

Clause 4: The intravascular medical device of clause 3, wherein the first period of time is defined by a release rate of the therapeutic agent.

Clause 5: The intravascular medical device of clause 3 or 4, wherein the first period of time is greater than two weeks and less than nine months.

Clause 6: The intravascular medical device of clause 4, wherein the release rate of the therapeutic agent is configured to reduce an amount of inflammation at the denervation target site over the first period of time.

Clause 7: The intravascular medical device any one of clauses 3 to 6, wherein the therapeutic agent is configured to inhibit growth of the portion of smooth muscle cells of the vessel over a second period of time following the first period of time.

Clause 8: The intravascular medical device of clause 7, wherein the second period of time is defined by at least one of a release rate or a loading of the therapeutic agent.

Clause 9: The intravascular medical device of clause 7 or 8, wherein the second period of time is greater than two weeks and less than nine months.

Clause 10: The intravascular medical device of any one of clauses 7 to 9, wherein the amount of the therapeutic agent is configured to remove the portion of smooth muscle cells sufficient to allow a lumen of the vessel to dilate via blood pressure over at least one of the first period of time or the second period of time, and wherein the amount of the therapeutic agent is configured to allow the vessel to heal with the dilated lumen and with a reduced amount of inflammation over the second time period.

Clause 11: The intravascular medical device of any one of clauses 1 to 10, wherein the therapeutic agent comprises a cytotoxin.

Clause 12: The intravascular medical device of any one of clauses 1 to 11, wherein the therapeutic agent comprises at least one of a chemotherapeutic agent, chemical agent, autoinflammatory agent, neurotoxin, or general toxin.

Clause 13: The intravascular medical device of any one of clauses 1 to 12, wherein the expandable member comprises a balloon.

Clause 14: The intravascular medical device of clause 13, wherein the balloon comprises a semi-compliant balloon configured to provide contact between the therapeutic agent disposed on the outer surface of the balloon and an inner surface of the renal vessel when expanded to deliver the therapeutic agent to the inner surface of the renal vessel.

Clause 15: The intravascular medical device of any one of clauses 1 to 14, wherein the therapeutic agent is configured to, at least one of: coat the inner surface of the vessel in the denervation target site, or be absorbed by the tissue in the denervation target site.

Clause 16: The intravascular medical device of any one of clauses 1 to 15, wherein the vessel comprises a renal vessel, and wherein the denervation therapy comprises renal denervation therapy.

Clause 17: The intravascular medical device of any one of clauses 1 to 16, wherein the denervation therapy comprises at least one of delivery of cold therapy, delivery of heat therapy, delivery of radiofrequency energy, delivery of ultrasound energy, or delivery of a chemical agent.

Clause 18: The intravascular medical device of any one of clauses 1 to 17, wherein the expandable member includes a blade configured to cut a portion of the vessel to a first depth from the inner surface of the vessel, and wherein the expandable member is configured to deliver at least a portion of the amount of the therapeutic agent to tissue at least one of adjacent the cut portion of the vessel or within the cut portion of the vessel.

Clause 19: The intravascular medical device of any one of clauses 1 to 18, wherein the expandable member is a first expandable member, the intravascular medical device further comprising a second expandable member configured to dilate the vessel at the denervation target site before expanding the first expandable member.

Clause 20: A method, comprising: delivering denervation therapy to a tissue adjacent to a vessel of a patient at a denervation target site; and after delivering the denervation therapy, expanding an expandable member proximate to the tissue to contact a therapeutic agent disposed on an outer surface of the expandable member to an inner surface of the vessel and deliver the therapeutic agent to the vessel.

Clause 21: The method of clause 20, further comprising positioning a distal portion of an implantable medical device in the vessel, wherein the distal portion of the implantable medical device comprises: one or more therapeutic elements configured to deliver the denervation therapy to the tissue; and the expandable member.

Clause 22: The method of clause 20 or 21, further comprising: positioning a distal portion of a neuromodulation catheter in the vessel, wherein the neuromodulation catheter is configured to deliver the denervation therapy to the tissue; and positioning the expandable member proximate to the tissue.

Clause 23: The method of any one of clauses 20 to 22, wherein the therapeutic agent is configured to remove a portion of smooth muscle cells of the vessel over a first period of time.

Clause 24: The method of clause 23, wherein the first period of time is greater than two weeks and less than nine months.

Clause 25: The method of clause 23 or 24, wherein the first period of time is defined by at least an amount of the therapeutic agent disposed on the outer surface of the expandable member.

Clause 26: The method of clause 25, wherein the amount of the therapeutic agent is configured to reduce an amount of inflammation at the denervation target site over the first period of time.

Clause 27: The method of any one of clauses 23 to 26, wherein the therapeutic agent is configured to inhibit growth of the portion of smooth muscle cells of the vessel over a second period of time following the first period of time.

Clause 28: The method of clause 27, wherein the second period of time is greater than two weeks and less than nine months.

Clause 29: The method of clause 27 or 28, wherein the second period of time is defined by at least the amount of the therapeutic agent disposed on the outer surface of the expandable member.

Clause 30: The method of any one of clauses 27 to 29, wherein the amount of the therapeutic agent is configured to remove the portion of smooth muscle cells sufficient to allow a lumen of the vessel to dilate via blood pressure over at least one of the first period of time or the second period of time, and wherein the amount of the therapeutic agent is configured to allow the vessel to heal with the dilated lumen and with a reduced amount of inflammation over the second time period.

Clause 31: The method of any one of clauses 20 to 30, wherein the therapeutic agent comprises a cytotoxin.

Clause 32: The method of any one of clauses 20 to 31, wherein the therapeutic agent comprises at least one of a chemotherapeutic agent, chemical agent, autoinflammatory agent, neurotoxin, or general toxin.

Clause 33: The method of any one of clauses 20 to 32, wherein the expandable member comprises a balloon.

Clause 34: The method of clause 33, wherein the balloon is a semi-compliant balloon configured to provide contact between the therapeutic agent disposed on the outer surface of the balloon and an inner surface of the renal vessel when expanded to deliver the therapeutic agent to the inner surface of the renal vessel.

Clause 35: The method of any one of clauses 20 to 34, wherein the therapeutic agent is configured to at least one of: coat the inner surface of the vessel in the denervation target site, or be absorbed by the tissue in the denervation target site.

Clause 36: The method of any one of clauses 20 to 35, wherein the vessel comprises a renal vessel, and wherein the denervation therapy comprises renal denervation therapy.

Clause 37: The method of any one of clauses 20 to 36, wherein the denervation therapy comprises at least one of delivery of cold therapy, delivery of heat therapy, delivery of radiofrequency energy, delivery of ultrasound energy, or delivery of a chemical agent.

Clause 38: The method of any one of clauses 20 to 37, wherein the expandable member includes a blade configured to cut a portion of the vessel to a first depth from the inner surface of the vessel, wherein the expandable member is configured to deliver at least a portion of the amount of the therapeutic agent to the tissue at least one of adjacent the cut portion of the vessel or within the cut portion of the vessel.

Clause 39: The method of any one of clauses 20 to 38, wherein the expandable member is a first expandable member, the intravascular medical device further comprising a second expandable member configured to dilate the vessel at the denervation target site before expanding the first expandable member.

Clause 40: An intravascular medical device comprising: an elongated member configured to be navigated through vasculature of a patient to a target treatment site in a vessel of the patient; one or more therapeutic elements positioned at a distal end of the elongated member, wherein the one or more therapeutic elements are configured to deliver neuromodulation therapy to the target treatment site; and an expandable member configured to be delivered to the target treatment site via the elongated member, wherein the expandable member is configured to deliver a therapeutic agent disposed on an outer surface of the expandable member to an inner surface of the vessel when expanded.

Clause 41: A method, comprising: expanding an expandable member proximate to tissue adjacent to a vessel of a patient at a target treatment site, wherein expanding the expandable member causes an outer surface of the expandable member to contact a therapeutic agent disposed on an outer surface of the expandable member to an inner surface of the vessel at the target treatment site and to deliver the therapeutic agent to the inner surface of the vessel, and wherein the therapeutic agent is configured to remove a portion of smooth muscle cells of the vessel over a first period of time.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

1. An intravascular medical device comprising:
a neuromodulation catheter configured to be navigated through vasculature of a patient and deliver denervation therapy to tissue at a target treatment site of a vessel; and
an expandable member configured to deliver a therapeutic agent disposed on an outer surface of the expandable member to an inner surface of the vessel when the expandable member is expanded.

2. The intravascular medical device of claim 1, wherein the therapeutic agent is disposed in a delivery medium configured to:
adhere to a wall of the vessel; and
release the therapeutic agent over a period of time according to at least one of a release rate of the therapeutic agent from the delivery medium or a loading of the therapeutic agent in the delivery medium.

3. The intravascular medical device of claim 1, wherein the therapeutic agent is configured to remove a portion of smooth muscle cells of the vessel over a first period of time.

4. The intravascular medical device of claim 3, wherein the first period of time is defined by a release rate of the therapeutic agent; or wherein the first period of time is greater than two weeks and less than nine months.

5. The intravascular medical device of claim 4, wherein the release rate of the therapeutic agent is configured to reduce an amount of inflammation at the denervation target site over the first period of time.

6. The intravascular medical device of claim 3, wherein the therapeutic agent is configured to inhibit growth of the portion of smooth muscle cells of the vessel over a second period of time following the first period of time.

7. The intravascular medical device of claim 6, wherein the second period of time is defined by at least one of a release rate or a loading of the therapeutic agent; or wherein the second period of time is greater than two weeks and less than nine months.

8. The intravascular medical device of claim 6,
wherein the amount of the therapeutic agent is configured to remove the portion of smooth muscle cells sufficient to allow a lumen of the vessel to dilate via blood pressure over at least one of the first period of time or the second period of time, and
wherein the amount of the therapeutic agent is configured to allow the vessel to heal with the dilated lumen and with a reduced amount of inflammation over the second time period.

9. The intravascular medical device of claim 1, wherein the therapeutic agent comprises a cytotoxin.

10. The intravascular medical device of claim 1, wherein the therapeutic agent comprises at least one of a chemotherapeutic agent, chemical agent, autoinflammatory agent, neurotoxin, or general toxin.

11. The intravascular medical device of claim 1, wherein the expandable member comprises a balloon.

12. The intravascular medical device of claim 11, wherein the balloon comprises a semi-compliant balloon configured to provide contact between the therapeutic agent disposed on the outer surface of the balloon and an inner surface of the renal vessel when expanded to deliver the therapeutic agent to the inner surface of the renal vessel.

13. The intravascular medical device of claim 1, wherein the therapeutic agent is configured to, at least one of:
coat the inner surface of the vessel in the denervation target site, or
be absorbed by the tissue in the denervation target site.

14. The intravascular medical device of claim 1,
wherein the vessel comprises a renal vessel, and
wherein the denervation therapy comprises renal denervation therapy.

15. The intravascular medical device of claim 1, wherein the denervation therapy comprises at least one of delivery of cold therapy, delivery of heat therapy, delivery of radiofrequency energy, delivery of ultrasound energy, or delivery of a chemical agent.

16. The intravascular medical device of claim 1,
wherein the expandable member includes a blade configured to cut a portion of the vessel to a first depth from the inner surface of the vessel, and
wherein the expandable member is configured to deliver at least a portion of the amount of the therapeutic agent to tissue at least one of adjacent the cut portion of the vessel or within the cut portion of the vessel.

17. The intravascular medical device of claim 1, wherein the expandable member is a first expandable member, the intravascular medical device further comprising a second expandable member configured to dilate the vessel at the denervation target site before expanding the first expandable member.

18. A method, comprising:
delivering denervation therapy to a tissue adjacent to a vessel of a patient at a denervation target site; and
after delivering the denervation therapy, expanding an expandable member proximate to the tissue to contact a therapeutic agent disposed on an outer surface of the expandable member to an inner surface of the vessel and deliver the therapeutic agent to the vessel.

19. The method of claim 18, further comprising positioning a distal portion of an implantable medical device in the vessel, wherein the distal portion of the implantable medical device comprises:
one or more therapeutic elements configured to deliver the denervation therapy to the tissue; and
the expandable member.

20. The method of claim 18, further comprising:
positioning a distal portion of a neuromodulation catheter in the vessel, wherein the neuromodulation catheter is configured to deliver the denervation therapy to the tissue; and
positioning the expandable member proximate to the tissue.
